(19) United States
(12) Patent Application Publication
(10) Pub. No.: US 2009/0044803 A1
(43) Pub. Date: Feb. 19, 2009

(54) Anaesthesia machine simulator

(76) Inventor: Javier Garcia Fernandez, Madrid (ES)

Correspondence Address: COLLARD & ROE, P.C., 1077 NORTHERN BOULEVARD, ROSLYN, NY, 11576, US

(21) Appl. No.: 12/228,712

(22) Filed: Aug. 15, 2008

Related U.S. Application Data
(60) Provisional application No. 60/965,081, filed on Aug. 17, 2007.

(51) Int. Cl. G09B 23/28; A61M 16/01

(52) U.S. Cl. 128/203.14; 434/262

(57) ABSTRACT

An anaesthesia simulator includes a sealed container, a gas input device that introduces gases into the sealed container whereto it is connected, a gas output and return device wherefrom gases exit the sealed container whereto it is connected, and a pressure generator connected to the sealed container which exerts pressure inside the sealed container.

This invention relates to an anaesthesia machine simulator which primarily allows anaesthesiologists to have better knowledge of the elements and parameters that govern a standard anaesthesia workstation. Moreover, this machine allows for the reproduction of the different critical situations which may arise during patient ventilation, so that anaesthesiologists are able to handle them in the most suitable manner for the patient.

### PRIOR STATE OF THE ART

Current anaesthesia machines have significantly evolved since 1903, when Harcourt used unidirectional valves to apply chloroform and provided the supply thereof to the patient by applying heat in order to increase its vaporisation. Between 1910 and 1930, scientists revolutionised the design of anaesthesia machines, which, from the 1930s, began to have characteristics very similar to present-day ones.

Anaesthesia machines are precision equipment endowed with mechanical, engineering and electronic details designed to ensure an exact, predictable volume of gas. Anaesthesia equipment has four important characteristics: a source of O₂ and a system to eliminate CO₂, a source of anaesthetic liquids or gases, and an inhalation system, which requires cylinders and their yokes, adjustment valves, flow metres, pressure metres and other systems designed to administer the anaesthetic mixture to the patient's respiratory tract.

One of the basic tasks of anaesthesiologists is to become familiar with anaesthesia machines, which requires not only being familiar with their operation, but ensuring that the main characteristics of their components comply with the safety standards published by the American National Standard Institute in standard Z 79.8. This tool allows specialists to choose and combine measured gases, vaporise exact volumes of anaesthetic gases and, therefore, administer controlled concentrations of the anaesthetic mixture through the respiratory tract.

However, becoming familiar with anaesthesia machines is done in a very superficial manner by most anaesthesiologists, who usually do not have in-depth knowledge about the machines they use, due to the complexity thereof.

Currently, anaesthesia machines are composed, on the one hand, of a ventilator designed with a circular circuit in order to utilise the gases expired by the patient and, on the other hand, a haemodynamic and respiratory monitoring assembly in order to control the patient under anaesthesia in the operating room.

Ventilators designed with a circular circuit are completely different from those used for patient ventilation outside the operating room, in critical care units, which are always open-circuit ventilators. In every breath, the open circuit always takes in fresh gases in order to ventilate the patient and, in the expiratory phase, the patient expels all the gases used to the outside. On the other hand, circular circuits allow anaesthesiologists to utilise the gases expired by the patients, once the CO2 is eliminated, and re-use them to ventilate them over and over again. This leads to savings in economic and environmental costs, since it reduces the consumption and release of anaesthetic gases. This type of ventilation, which, as default, should be performed using the low-flow dosing technique, is called controlled mechanical ventilation.

Therefore, contrary to what occurs with open-circuit ventilators (critical care), circular-circuit ventilators must be understood in depth so that no problems arise when ventilating patients under special circumstances (severely obese patients, pregnant women, premature babies, healthy newborns, patients with laparoscopy, etc.), and particularly children (less than 10 kg in weight), wherein clinical incidents due to inadequate use of anaesthesia machines is 1:10,000, barotrauma, hypoxaemia and hypercapnia being the complications with the highest reported incidence, which tend to cause serious, permanent neurological injuries, and even the death of patients due to anaesthetic reasons.

On the other hand, circular-circuit anaesthesia machines or stations are capable, as specified above, of utilising the anaesthetic gases expired by the patients in order to subsequently re-use them. In order to perform this ventilation efficiently and take advantage of circular-cycle anaesthesia stations, anaesthesiologists must specify the minimum metabolic oxygen consumption which the patient needs (generally between 200 and 300 ml of O₂ per minute—low flow—), and simultaneously increase the concentration of anaesthetic gas.

In this manner, the total volume of anaesthetic gas that reaches the patient is the same that they would receive if the O₂ flow were greater and the concentration of anaesthetic gas were lower (high flow), as is the case with open circuits. Surprisingly, when anaesthesiologists who use circular-circuit anaesthesia machines are asked about the concentrations of anaesthetic gas and the O₂ flows which they supply to the patients, one concludes that, in a very high percentage of cases, surgeries are performed with high-flow dosing. As a consequence, when the gas dosed at high flows mixes with the gas expired by the patients, there is an increase in gas concentration and pressure, which must be reduced using an overflow valve, and the anaesthetic gases are not economised.

The main difference between a circular circuit and an open circuit is that the circular circuit must have the following components and parameters which the open circuit lacks:
Patient circuit with an inspiratory branch and an expiratory branch, and a “Y”-part to be connected to the patient.
Unidirectional valves (inspiratory and expiratory).
Fresh gas flow entry point.
Vaporiser designed to administer the anaesthetic gases.
An internal circuit volume.
A gas reservoir (bag, concertina bellows, etc.).
Overflow valve or pop-off valve.
APL or pressure release opening valve.
A CO₂ canister or absorber.
Flow generator independent from the gas input (concertina bellows, piston or turbine).

These components cause the circular circuit to have a series of elements and parameters which must also be considered when operating this type of anaesthesia workstations:
Time constant
Compliance (volume/pressure)
Compliance or distensibility compensation systems.
Fresh gas flow utilisation rate
Leaks
Low-flow dosing

All these specific characteristics of circular circuits, which open circuits do not have, cause anaesthesiologists to be prone to having more clinical ventilation problems than other specialists who use open-circuit ventilation. Thus, if an open circuit is used for ventilation, it is not necessary to be familiar with the ventilator's internal design, since they do not generate adverse circumstances in clinical practise. However, given the different designs of different circular circuits, anaesthesiologists who are not familiar with and do not perfectly understand all the characteristics of the anaesthesia station they are using, may have complications when ventilating patients, particularly under special circumstances.

### BRIEF DESCRIPTION OF THE INVENTION

The author of this invention has developed a circular-circuit anaesthesia simulator which reproduces each and every part that makes up an anaesthesia machine. This simulator allows for the reproduction of different clinical situations, primarily adverse ones, which may arise during the process of ventilating patients, and helps anaesthesia machine users who carry it out.

### DEFINITIONS

The terms “anaesthesia table, machine, station, ventilator or equipment” refer to the set of elements used to administer fresh anaesthetic gases to patients during anaesthesia, in both spontaneous and controlled ventilation.

The term “controlled ventilation” refers to situations wherein patients are ventilated in accordance with the control variables pre-set by the anaesthesia machine operator. In the absence of inspiratory effort by the patient, the ventilator provides controlled respiration. This ventilation is called “mechanical” when it is performed using the mechanical pressure generation system known as piston, bellows, concertina bellows, etc., and “manual” when it is performed using the manual pressure generation system.

The term “anaesthesia simulator” refers to a machine that is capable of reproducing the different situations which arise with an anaesthesia workstation during the ventilation process, as well as the tests or checks that these machines perform. Consequently, this device does not need to have all the elements that make up circular-circuit anaesthesia machines and cannot be used to ventilate patients.

In the description, the term “pressure generation system” refers to a bellows, piston, concertina bellows, turbine or any other type of device that allows for the generation of a positive pressure in the anaesthetic circuit, so as to favour the input of gas into the inspiratory branch.

In the description, the term “canister or filter” refers to a container filled with soda lime or barium lime, the purpose whereof is to absorb the CO2 from the patient's expirations (“expired gas”) so that the latter does not inspire them in the next inspiration.

The term “vaporiser” refers to machines the function whereof is to produce vaporisation of volatile liquids within a regulable concentration. In other words, they are in charge of controlling the concentration of anaesthetic gases that is supplied to the patient jointly with the oxygen.

The term “pop-off valve” or overflow valve refers to devices that eliminate the excess pressure generated by the excess gas present in the circular circuit. This term is closely related to the “fresh gas flow utilisation rate”, which is explained further below.

The term “internal circuit volume” refers to the sum of the volumes of all the anaesthesia machine's internal components. This internal volume determines the speed wherewith the gas and the expired gas mix, and, in the simulator, it is represented, jointly with the gas reservoir, by the container.

In the description, the term “gas reservoir” refers to a container designed to collect the “gas” flow that penetrates into the anaesthetic circuit and is mixed with the expired gas, in order to be propelled to the patient by compression. This gas reservoir is concealed in the interior of anaesthesia stations and, in the simulator, is represented by the container.

The term “time constant” refers to the time which the anaesthesia machine takes to fill up with or empty out the new gases. In open circuits, this constant is practically null, because, since there is not a significant internal circuit volume, the time elapsed from the moment the gas pressure is exerted until it reaches the patient is insignificant. In circular circuits, depending on how they are built, this constant is more or less high.

The term “APL valve” (adjustable pressure-limiting valve) refers to a valve the function whereof is to regulate the pressure supplied to the circular circuit through the manual pressure generation system. This valve is usually confused in the literature with the pop-off valve.

The term “tidal or current volume” is the volume of air that enters the patient in each inspiration. If we consider that a person makes a given number of inspirations per minute, this figure makes it possible to determine the volume of air inspired per minute (“minute volume”). This minute volume is approximately 200 ml/kg for children under 10 kilos in weight and 100 ml/kg for children over 10 kilos and for adults.

The term “compliance of the anaesthesia machine” refers to the compressible volume that remains compressed inside the anaesthesia machine for every cm of H₂O of positive pressure that is generated in mechanical ventilation. This volume is retained inside the anaesthesia machine and, if it is not compensated, subtracts and reduces the patients' current volume.

The term “compressible volume” refers to the property of gases whereby their volume is reduced when they are subject to a given pressure; this concept is governed by Boyle's gas compressibility Law, which states that, when “a gas is subjected to a given pressure, it acquires a new, lower volume, and that the product of the initial pressure by the initial volume is equal to the product of the final pressure by the final volume (P×V=P′×V′)”. The compressible volume increases the greater the internal volume of the anaesthesia machine and the circuit nozzles and the higher the maximum pressure achieved during positive-pressure mechanical ventilation. In order to determine it, one must place a known volume of gas and measure the pressure with the manometer. The volume divided by the pressure gives the circuit compliance, which is used to calculate the volume of gas that must be introduced into the piston.

The term “compliance compensation systems of the anaesthesia machine” refers to systems designed to minimise the effect explained above. Depending on how effective they are, more or less current volume is lost in each patient ventilation.

The term “fresh gas flow utilisation rate” expresses, as a percentage, the volume of the total fresh gas administered to the anaesthesia machine that ends up reaching the patient. Due to the different circular circuit designs, not all of them utilise 100% of the fresh gases that enter therein, but a part of them are expelled into the environment even before reaching the patient. This situation never arises in open-circuit ventilators, which always have a fresh gas flow utilisation rate of 100%.

The term “machine leaks” refers to the gas losses that take place along the anaesthesia machine's circular circuit through the different connections between the components thereof.

The term “patient leaks” refers to the gas losses that take place when endotracheal tubes without pneumoplugging or supraglottic devices are used for the mechanical ventilation of patients; under these circumstances, gas leaks may occur between the supraglottic device or the tube and the patient's glottis or trachea; these leaks inside the patient are variable and also subtract volume for the next circular-circuit ventilation. Throughout the description, the terms “machine leaks” and “patient leaks” will be generally called “leaks”.

The term “low-flow dosing” refers to the dosing method that may and should be used as default in circular-circuit anaesthesia machines. This system consists of supplying the anaesthesia machine with the minimum fresh gas flow to cover the patient's oxygen consumption (minimum metabolic consumption of O₂) plus the total leaks, and thus allows achieving considerable cost savings by saving anaesthetic gases.

The term “Mapleson system” refers to a manual continuous-flow ventilation system that is incorporated into anaesthesia stations. These circuits were designed to perform spontaneous, manual ventilation without the need for any anaesthesia machine, starting solely from a continuous and constant source of fresh gas. These circuits are optional in anaesthesia machines but highly recommendable, since they allow ventilating the patient if the anaesthesia machine ceases to operate or fails; using these circuits we may even be able to continue to administer anaesthetic gases.

### BRIEF DESCRIPTION OF THE FIGURES

FIG. 1. This figure shows an anaesthesia machine or station.

FIG. 2. This figure shows a full panoramic view of the anaesthesia simulator with the main elements that compose it.

FIG. 3. This figure shows the gas output and return system.

FIG. 4. This figure shows the overflow elimination system.

FIG. 5. This figure shows the manual ventilation system.

### DETAILED DESCRIPTION OF THE INVENTION

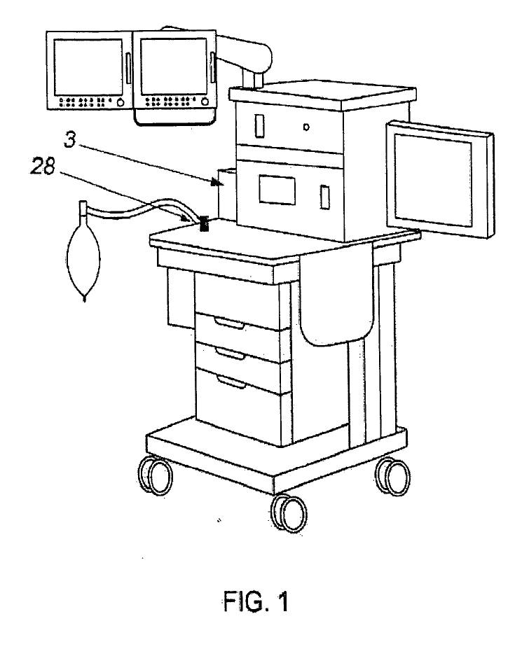

Due to the large differences between open-circuit critical-care ventilators and the current circular-circuit anaesthesia ventilators, when they are turned on, anaesthesia workstations (FIG. 1) require a series of preliminary checks to verify that they operate correctly and they supply information to the anaesthesiologist, who must be able to interpret it in order to prevent ventilation problems during the surgery.

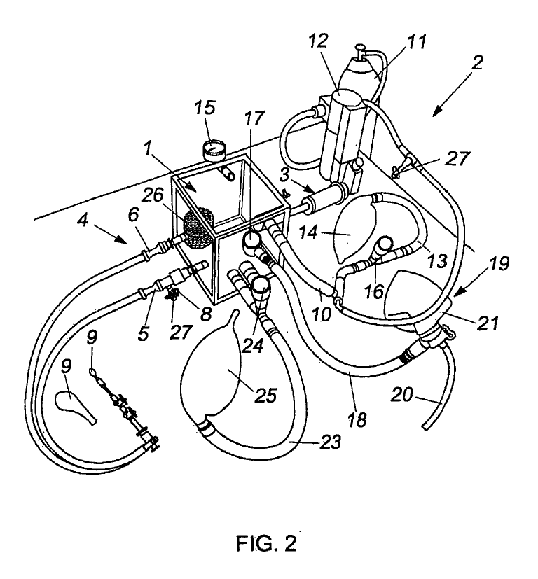

The author of this invention has developed a circular-circuit anaesthesia simulator (FIG. 2) that reproduces each and every part of an anaesthesia machine. Moreover, this simulator allows for the reproduction of different, primarily adverse, clinical situations which may arise during the patient ventilation process. Similarly, this device helps anaesthesiologists to better understand the elements, the operation and the variables that govern anaesthesia machines, thus allowing them to know, at all times, the problems which may arise and how to solve them, in order to prevent ventilation problems when the patients are under anaesthesia.

Below, we will illustrate how the anaesthesia simulator helps anaesthesiologists to become familiar with all the elements that compose an anaesthesia machine's circular circuit, their location and how they are interconnected, such that specialists may better know the machines that they use. Moreover, the simulator allows for a better understanding of those parameters that are difficult to understand, and which are inherent to these machines. This deeper knowledge will not only allow for a more adequate handling of anaesthesia stations, leading to cost savings, but will also prevent adverse clinical situations during anaesthesia processes which generate avoidable damages to the patients.

Thus, a first aspect of this invention relates to an anaesthesia simulator (hereinafter, the simulator —FIG. 2—) that comprises a sealed container (1), preferably transparent, and, more preferably, with a variable volume, whereto the elements selected from the group comprising the following are connected:
A gas input device or system (2) that introduces gases, preferably O₂, into the sealed container (1).
A system or system capable of generating a flow and pressure (3) inside the sealed container (1) (“mechanical flow-generation system”). This system, which comprises flow- or pressure-generation means, is capable of pressing the gas introduced by the gas input system (2), in order to direct it to the gas output and return system (4). Preferably, the flow-generation means may comprise, without any limitation whatsoever, a piston, a turbine, a bellows, a bag, a syringe or a concertina bellows.
A gas output and return device or system (4) (“patient circuit”) wherethrough the gases pushed by the mechanical flow-generation system penetrate (3), in order to be returned once again to the sealed container (1) when the pressure exerted by the system (3) ceases.

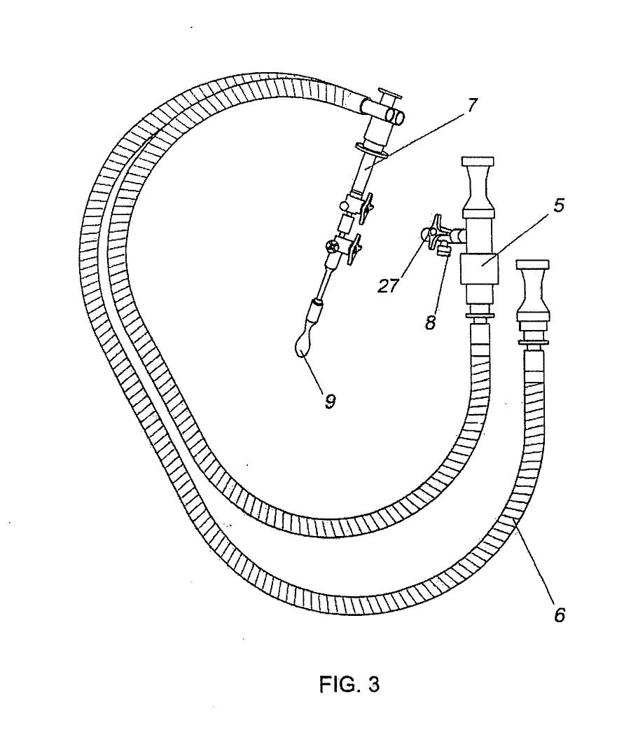

In a preferred embodiment, the patient circuit or gas output and return device (4) comprises (FIG. 3):
An inspiratory or gas output branch (5) that contains a unidirectional valve which allows for the input of gas from the sealed container (1), but prevents the return thereof through the same route. In a preferred embodiment, this inspiratory branch (5) would have an auxiliary gas input (8) that allows for the reproduction of a special type of anaesthesia machine (see example 3).
An expiratory branch (6) connected to the inspiratory branch (5), which contains a unidirectional valve that prevents the input of gas from the sealed container (1), and allows for the output of gas from the inspiratory branch (5). In an even more preferred embodiment, a CO₂ canister or filter (26) is connected at the outlet of the expiratory branch.

In another preferred embodiment of this aspect of the invention, the connection between the inspiratory and expiratory branches is performed through a conduit (7), which would simulate the patient or the respiratory tract thereof (“patient simulator”). Preferably, the conduit (7) is connected to a valve (27) which allows for the opening and closing thereof, thus allowing for the total or partial output of the gas that penetrates through the inspiratory branch, in order to simulate variable-magnitude patient-leak situations. Moreover, this valve (27) may be used as a gas input in order to simulate gas capture processes.

In practise, the free end of the patient simulator (conduit (7)) may additionally be connected to an inflatable element (9) (FIG. 3) that acts as the patient's lungs (“lung simulator”), increasing its size when pressure is exerted inside the circuit and decreasing its size when said pressure ceases or leaks are simulated.

In a preferred embodiment of this aspect of the invention, the gas input device (2) would consist of an input conduit (10) connected to a supply source of O₂ or any other gas (“the source”) (11). In an even more preferred embodiment, this device (2) would comprise an input conduit (10) connected to the source (11) and to a vaporiser (12). In an even more preferred embodiment, the input conduit (10) is connected or branches off to an auxiliary conduit (13) that has a bag (14), or any other type of element that allows for the generation of pressure, coupled to the end thereof, and along which there is an APL valve (16) or any other type of valve capable of regulating the pressure supplied by the bag (14). This system, which comprises the elements (13 and 14) and which, in parallel to the piston, bellows, etc, makes it possible to exert pressure inside the circuit, is known in the field of anaesthesia as the “Mapleson auxiliary circuit”.

In an even more preferred embodiment of this aspect of the invention, the simulator is connected, preferably on sealed container 1, to a manometer (15) that allows for the measurement of the pressure inside the circuit.

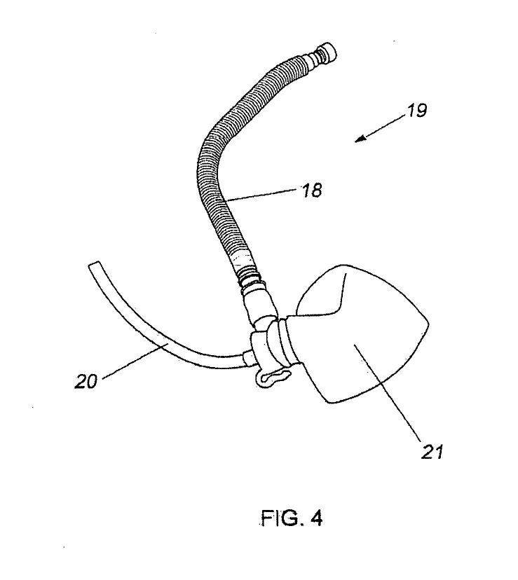

In an even more preferred embodiment of this aspect of the invention, the simulator comprises an overflow or excess pressure elimination device (19) (FIG. 4), which comprises a pop-off or overflow valve (17). Preferably, said valve is connected to an overflow or overpressure elimination conduit (18) with the excess gas outlet at the end thereof, which is connected to means designed for the extraction or evacuation of the excess gases introduced into the circuit. Said extraction system preferably comprises a nozzle (20) connected to a reservoir bag (21). This reservoir bag could additionally comprise a connector to communicate the interior thereof with the environment, and another connector which may be connected to an external vacuum inlet.

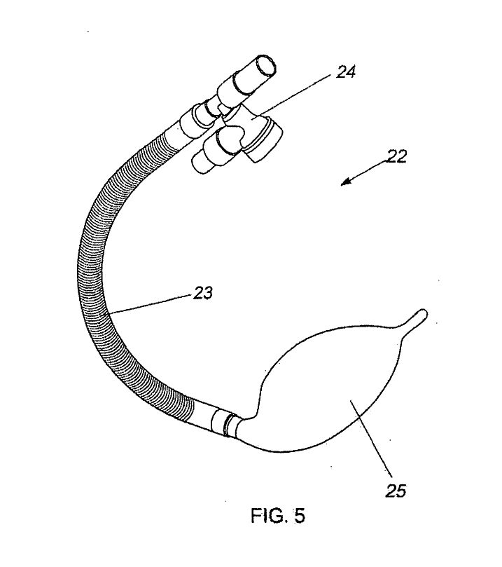

In another preferred embodiment of this aspect of the invention, the sealed container (1) is connected to a second device (22) (FIG. 5) capable of exerting a positive pressure in the interior thereof (“manual pressure generation system”). In a preferred embodiment, this system (22) would be composed of at least: one conduit (23) along which an APL valve (24), or any other type of valve capable of regulating the pressure of the air passing through the conduit (23), is connected, to be transmitted to the patient circuit (3), and a manual ventilation bag or any other pressure-exerting means (25) connected to the free end of the conduit (23).

In an even more preferred embodiment of this aspect of the invention, the simulator would be connected, along its circuit, to at least one valve (27) designed to open and close the conduits or the sealed container, in order to simulate leaks in the machine or in the patient circuit, in addition to unidirectional valves that allow for the direction of the gas flows.

Finally, we should state that some of the elements that are to form a part of the simulator may be replaced with non-functional elements that imitate the real ones; this is the case with the canister, the vaporiser or the pop-off and APL valves. This is due to the fact that these elements are not essential for the simulator, since the function of the latter is not to ventilate patients.

### DETAILED EXPLANATION OF THE EMBODIMENTS

### Example 1

### Leak Test

When an anaesthesia machine is fully sealed, that is, when there are no leaks through any of the connections between its components, the pressure exerted in the interior thereof remains constant with time.

In order to perform this check, the anaesthesia machine introduces a known pressure into the circuit through the piston (3), as a standard rule, 30 cm H₂O, and, once the machine is pressurised to this pressure, it interrupts the flow and calculates the pressure loss that takes place in one minute, thus calculating the leaks in the anaesthesia machine in one minute. What other machines do is to calculate the gas flow which they need to continue supplying during that minute in order for the pressure to remain at 30 cm H₂O for one minute, leading to the same calculation.

This same test may be easily simulated in the simulator by allowing the input of gases through the flow generator (2) towards the container (1), exerting pressure with the piston (3) and measuring the pressure variations in the circuit with the manometer (15). If the container and the interconnections between the simulator elements are sealed, no leaks will occur (constant pressure in the manometer), although these may be simulated from the valves (27). Thus, a process that is difficult to understand when explained using a standard anaesthesia machine, where what the machine does may not be visualised, becomes very simple to understand. In practise, these checks are performed by the machine operators, who only need to repeat a series of pre-established steps, without really knowing the implications or basics thereof.

### Example 2

### Compressible Volume

In an open circuit, the gas pressure supplied to a patient is directly transmitted thereto. On the other hand, in a circular circuit, the volume of gas contained in the interior thereof is capable of compressing when a pressure is exerted on the piston (3), just like when a pressure is exerted on a syringe piston, whilst the open end is kept blocked.

In order to perform this check, the anaesthesia machine introduces a known volume of air into the circuit through the piston, concertina bellows, turbine or other flow generator, which translates into an increase in the internal circuit pressure that is measured by the manometer. If the pressure remains constant, the machine calculates, from the volume and the pressure, the circuit compliance (volume/pressure), which in most cases ranges between 5 and 7 (ml/cm H2O), depending on each machine's internal volume. If this compliance coincides with that which corresponds to the machine on the basis of its internal volume, this suggests that there are no leaks and the machine may continue to operate safely. Otherwise, the compliance would increase, since the pressure decreases, the value would not coincide with that expected for the machine and this would indicate that it is out of range and not safe to be used. This same test may be performed using the elements that compose the simulator, making the process very easy to understand for the anaesthesia machine operator, particularly if the gas used is not colourless.

### Example 3

### Time Constant

The time constant is the time which a given container takes to fill up or empty out by 63%, and is an exponential process. Thus, 63% of filling up or emptying out of the container will take place in one time constant, 86% will take place in two time constants, and 95% will take place in three time constants.

The time constant of an anaesthesia machine depends on the internal circuit volume and the fresh gas flow used, minus the circuit leaks. The system's efficiency or fresh gas flow utilisation percentage also affect the time constant.

Currently, there are different ways to introduce the fresh gas flow into the anaesthesia machine; (i) one of these systems supplies the air through the input conduit (10), jointly with the anaesthesia gases, coming from the vaporiser (13) and mixed with O₂ coming from the source (11). This fresh gas is taken to a reservoir chamber (represented by the sealed container (1) in the simulator), in order to be pushed by the concertina bellows (3). (ii) The other system also introduces the anaesthesia gas through the input conduit (10), but the fresh gas enters directly at the inspiratory branch (5). Thus, the first system will have a much higher time constant than the second system. In order to reproduce the second of the above-mentioned systems (ii), it would suffice to disconnect the auxiliary conduit (13) from the input conduit (10), and couple the free end thereof to the gas input (8) of the inspiratory branch (5).

In situations of hypoxia (lack of O₂), hypercapmia (excess of CO₂), or bronchospasms (closing of the bronchi), where, if the patient remains without O₂ for more than 3 minutes, the brain damage is irreversible, anaesthesiologists quickly resort, in most cases, to the manual or Mapleson ventilation system (which is independent from the machine's internal circuit) in order to recover the patients as soon as possible and supply them with the O₂ that they need. This method, which makes sense when the first system mentioned in the preceding paragraph is used (i), is inappropriate when the second system (ii) is used (reduced efficiency in patient care), due to the fact that the anaesthesiologists devote their efforts to ventilating the patients, instead of administering the drugs they immediately need.

Therefore, adequate knowledge of the typology and design of the anaesthesia machine that they are using would help to prevent this type of situations.

### Example 4

### Pop-Off or Overflow Valve

Overflow valves (17) eliminate the excess fresh gas flow in the circular circuit, in order to prevent that the excess pressure produced from being transmitted to the patients and cause barotrauma or rupture of the lungs due to pressure on the respiratory tract. These valves are also subject to checking when the anaesthesia machine is turned on.

Occasionally, the overflow valves (17) may become obstructed during a surgery and cause barotrauma in the patients, particularly those whose respiratory tract is not very elastic. This circumstance is more common when patients are anaesthetised at high flows.

In order to explain this situation in the simulator, gas is supplied at a high flow through the gas input (2) and, a few seconds later, by means of the piston (3), a volume of air similar to that which a patient would normally be supplied is supplied to the circuit. If everything operates correctly, the gas will enter through the inspiratory branch (5), inflate the inflatable element (9) and re-enter through the expiratory branch (6). Now, gas at the pressure specified in the beginning continues to enter through the gas input; when mixed with the expired gas, it would increase the pressure inside the container. If the overflow valve (17) operates correctly, it will be possible to observe the gas output therethrough, as well as the gas input through the inspiratory branch (5) towards the balloon (9).

This process will be even more noticeable if the gas is coloured. If, on the other hand, we perform the same operation, but the pop-off valve is somehow obstructed, the excess pressure would be quickly transmitted to the inflatable element (9), and might even break it. If, moreover, the patient is a newborn baby, a premature baby, a pregnant woman or has a respiratory tract that is not very flexible (lung with fibrosis, patient with laparoscopy, severe obesity or respiratory distress), the result may be lethal.

These simple experiments help anaesthesiologists to become more familiar with the anaesthesia machines that they use, thereby preventing these types of situations.

### Example 5

### Mapleson or Continuous-Flow Controlled Direct Ventilation System

Most anaesthesia machines have a Mapleson auxiliary circuit (elements 13, 14, 16), which may be optional, but in most cases is recommended for safety reasons, in case the anaesthesia machine's main circular circuit fails, to thus have an alternative to ventilate the patient.

However, many specialists do not understand the utility of using this Mapleson circuit, as opposed to the circular one, for manual ventilation in certain critical situations for patients, such as bronchospasms (closing of the bronchi) and desaturations (oxygen reduction in the blood). This is so important that, in some countries and hospitals, in order to reduce costs, it is requested that anaesthesia machines do not include this circuit, thus selling them without this accessory circuit.

With the anaesthesia simulator, it is very easy to visualise all the differences between manual ventilation with the anaesthesia machine's circular circuit, using the manual pressure generation system (22) and manual ventilation with the Mapleson circuit. Thus, it is possible to easily observe all the connections between both systems, and how the Mapleson circuit is fed by the fresh gas flow directly programmed by the anaesthesiologist, and how, On the other hand, the circular circuit is fed by the mixture between the fresh gas specified by the anaesthesiologist and the gas that the machine receives from the patient, which delays the time required to change the gas concentration received by the patient.

### Example 6

### Low-Flow Dosing

This is the main purpose wherefor circular circuits were designed in anaesthesia: savings in anaesthetic gases. The dosing method in open circuits is very easy, since the amount of fresh gases specified in the machine is what reaches the patient in each ventilation. However, in circular circuits the same thing does not occur, because, if we use low fresh gas flows, these mix with the gases that return from the patient, who is ventilated with the mixture of both types of gases in the next ventilation. Therefore, the concentration of anaesthetic gas that is specified in the fresh gas need not be the same that reaches the patient. This determines that low-flow dosing of gases in circular circuits is technically more complex and not easy to understand.

The proof of the limited knowledge about this type of systems is found in that a high percentage of anaesthesiologists use high flows when they use circular-circuit machines, when they should use low flows.

In order to visualise this process in the simulator, gas is supplied at a high flow through the gas input system (2) and, a few seconds later, through the piston (3), a volume of air similar to that which would normally be supplied to a patient is supplied to the circuit. If everything operates correctly, the gas in the container (1) will enter through the inspiratory branch (5), inflate the balloon (9) and re-enter through the expiratory branch (6) to the container (1), passing through this branch's unidirectional valve and through the canister (26), where the CO₂ would be trapped. Now, gas at the pressure specified in the beginning continues to enter through the gas input; when mixed with the expired gas, the pressure inside the container would increase. If the overflow valve (17) operates correctly, it will be possible to observe the gas output therethrough and a second gas input through the inspiratory branch (5), which ends up causing an increase in the balloon volume (9) once again.

In order to show the low-flow dosing technique, the same process described in the preceding paragraph would be used, but with low-flow dosing. The only difference that would be observed in this case is that no gas leaks take place through the overflow valve (17) during the second input of gas through the inspiratory branch (5) and, consequently, there would be no misutilisation of the anaesthetic gases.

### Example 7

### Manual Controlled Ventilation Through the Anaesthesia Machine

In the event of a problem with the anaesthesia machine's flow generator (2), anaesthesiologists may choose different systems to continue ventilating the patients. One of these systems is the Mapleson system, explained above, and the other consists of manual ventilation that incorporates the anaesthesia machine's circular circuit, which, in the simulator, has been called manual pressure generation system (22). Unlike the Mapleson system, this system utilises the machine's circular circuit.

From the simulator, it is easily observed that, using the bag (25), it is possible to exert a positive pressure in the circuit. This pressure is transmitted through the conduit (23), passing through the APL valve (24) that regulates and releases it, such that it finally pushes the gas in the container (1). This mechanism, like the others discussed, is difficult to understand when using a standard anaesthesia machine, where it is also possible to switch to the manual controlled ventilation system, generally by simply turning a lever (28) (FIG. 1). The simulator thus allows obtaining in-depth knowledge about what happens when switching from the mechanical controlled ventilation system to the manual one, particularly if coloured gases are used.

1: Anaesthesia simulator comprising:
a. a sealed container (1),
b. a gas input device (2) that introduces gases into the sealed container (1) whereto it is connected,
c. a gas output and return device (4) wherefrom gases exit the sealed container (1) whereto it is connected,
d. pressure-generation means (3) connected to the sealed container which exert pressure inside said sealed container (1).

2: Anaesthesia simulator, according claim 1, wherein the gas output and return device (4) comprises:
a. an inspiratory branch (5) connected to the sealed container (1), which includes a unidirectional valve that prevents the return of gases towards the sealed container (1),
b. an expiratory branch (6), connected to the inspiratory branch (5) and to the sealed container (1), which drives the gases that circulate through the inspiratory branch (5) towards the sealed container (1) and includes a unidirectional valve that prevents the input of gases from the sealed container (1).

3: Anaesthesia simulator, according to claim 1, wherein the inspiratory branch additionally comprises an auxiliary gas input (8) provided with a valve (27) that allows it to open and close.

4: Anaesthesia simulator, according to claim 1, wherein the gas input device (2) comprises a gas supply source (11) and an input conduit (10) which connects the source to the sealed container (1).

5: Anaesthesia simulator, according to claim 1, wherein the pressure-generation means (3) are selected from the group formed by a piston, a turbine, a bellows, a syringe or a concertina bellows.

6: Anaesthesia simulator, according to claim 1, wherein the input conduit (10) branches off or connects to an auxiliary conduit (13) associated with a bag (14) or any other type of means capable of generating pressure.

7: Anaesthesia simulator, according to claim 1, wherein the inspiratory branch (5) is connected to the expiratory branch (6) by means of a conduit (7) which comprises a valve (27) that regulates the output of gas from the inspiratory branch (3) towards the exterior and the sealed container (1) through the expiratory branch (4).

8: Anaesthesia simulator, according to claim 7, further comprising an inflatable element (9) connected to the free end of the conduit (7) in order to simulate the patient's lungs.

9: Anaesthesia simulator, according to claim 1, further comprising a manometer (15) that is connected to the sealed container (1) and measures the pressure in the interior thereof.

10: Anaesthesia simulator, according to claim 1, further comprising an overflow elimination device connected to the sealed container (1).

11: Anaesthesia simulator, according to claim 10, wherein the overflow elimination device comprises an overpressure valve (17) connected to the sealed container (1) and an overflow elimination conduit (18) which has gas extraction or evacuation devices (19) coupled to the end thereof.

12: Anaesthesia simulator, according to claim 1, further comprising a pressure-generation device (25) connected to the sealed container (1) through an input conduit, along which it is connected to an APL valve (24) that regulates the air pressure that is auxiliary introduced into the sealed container (1) with the device (25).

13: Anaesthesia simulator, according to claim 1, further comprising at least one valve (27) connected along the circuit thereof which allows for the release of pressure.
